(19) United States
(12) Patent Application Publication
(10) Pub. No.: US 2016/0008160 A1
(43) Pub. Date: Jan. 14, 2016

(54) Thermoformable laminate for orthopedic splints

(71) Applicant: Leah Rotter, Gardena, CA (US)

(72) Inventor: Leah Rotter, Gardena, CA (US)

(21) Appl. No.: 14/682,638

(22) Filed: Apr. 9, 2015

Related U.S. Application Data
(60) Provisional application No. 61/978,197, filed on Apr. 11, 2014.

(51) Int. Cl. A61F 5/058

(52) U.S. Cl. CPC A61F 5/05825; A61F 5/05858; A61F 5/0585

(57) ABSTRACT

A thermoformable laminate for use in making orthopedic splints is composed of a sheet of thermoformable splinting material having opposite sides and a covering layer applied to at least one of those sides as padding for more comfortable application and wear on a patient's limb. The covering layer may be applied to the thermoformable sheet by a fiber flocking process and is chosen to allow heating of the thermoformable sheet to a deformable state by means such as hot air blowers or immersion in hot water. The laminate may be cut with scissors or the like and formed to custom shapes resulting in ready-to-apply orthopedic splints after attachment of splint retaining straps.

This application claims priority to Provisional Patent Application No. 61/978,197 filed Apr. 11, 2014

### FIELD OF THE INVENTION

This invention pertains generally to the field of orthopedic splints which provide temporary support for limbs and joints compromised by disease, injury or aging, and more particularly relates to light weight laminate materials with integral padding for comfort and hygiene which can be cut and shaped in the field to make customized orthopedic splints.

### STATE OF THE PRIOR ART

Orthopedic splints are devices which bridge anatomical joints of a patient's limbs, such as knees, elbows and wrists, for the purpose of limiting mobility of the joint and/or providing support to injured or diseased joints.

It is known to make orthopedic splints using thermoformable plastics that can be heated to a deformable state, by immersion in hot water or with hot air guns, and then bent and shaped to fit a particular patient's anatomical shape. Also known are pre-fabricated splints having hinged or bendable cores of sheet metal or metal wire for the same purpose. In existing splints of this type the adjustable or deformable core is often inserted in a separate soft cover of washable fabric which provides a degree of cushioning or padding and which can be removed for laundering as needed. The removable covers may have attached straps which are wrapped about the patient's limb for fastening the splint in place.

A line of splints is sold by Lenjoy Medical Engineering of Gardena, Calif., under the mark Comfyprene, which does away with removable covers and instead has a bendable but substantially rigid splint frame made of a sheet of metal, such as cold rolled steel, contained and fully enclosed between foam plastic sheets bonded to each other to make a seam around the edges of the sheet metal splint frame, and further covered with and contained between opposite layers of resilient neoprene rubber bonded to the foamed plastic sheets. A layer of fabric is laminated to the outer surfaces of the neoprene layers such that splint retaining straps can be attached with hook-and-loop type fasteners, with the laminated fabric serving as one gender of the hook and loop pair. The metal frame of this splint product is not thermoformable and even if the metal frame were replaced with thermoformable plastic the neoprene covering insulates the stiff inner frame and as a practical matter would prevent sufficient heating of a thermoformable frame.

While the aforementioned splints are in wide use by physical therapists, a continuing need exists for a splint material having the characteristics of thermoformable splinting plastic sheet material, i.e. relatively low cost, lightweight sheet material which can be readily bent, shaped and cut, but in addition having integral padding which does not significantly impede the heating of the thermoformable material to a deformable state by immersion in hot water or with a hot air blower, and which can be cut as a laminate using hand scissors or the like so that a therapist may readily create custom splint shapes in the field.

### SUMMARY OF THE INVENTION

This invention is an orthopedic splinting laminate having a core of thermoformable splinting sheet material with opposite sides and a padding layer applied to at least one of the sides, where the padding layer is chosen for hygiene, comfort and protection from abrasion. The padding layer is further chosen to allow relatively quick heating of the core sheet to a thermoformable state by immersion in hot water or by application of heated air, as with a hot air blower or heat gun type device. A preferred padding layer is applied by flocking the surface of the core thermoformable sheet with cut fiber material. The flocking may be applied by a conventional flocking process.

The integrally padded splinting laminate according to this invention can be manufactured at relatively low cost by continuous flocking processes, including existing flocking processes, compared to costlier and more difficult traditional approaches for making orthopedic splints. The laminate of this invention can be made as standardized flat stock sheets for subsequent cutting to custom shapes and sizes. Alternatively, the laminate may be fabricated as precut flat splint shapes suitable for different kinds of splints, such as hand and wrist splints, knee splints and elbow splints, and which could still be cut down and shaped further in the field, if desired, to achieve optimal fits. The flocking may be applied to pre-cut splint shapes of thermoformable splinting plastic, not only to one or both sides of the pre-cut splint shape, but may be applied also to some or all edges of the thermoformable plastic between the opposite sides of the pre-cut plastic splint shape.

In another form of the invention, the padding layer is of a material adapted to make retentive engagement with a hook or loop type fastener, so that a retaining strap provided with a complementary hook or loop type fastener may be attached at any desired location of a splint cut from such laminate. For example, the padding layer may be a sheet of fabric adhesively applied to cover one or both sides of the core thermoformable splinting sheet material. One presently preferred fabric is so-called “headliner” fabric of the type used in automotive interiors. Such headliner fabric is currently used to make covers for orthopedic splints because it has the property of serving as a loop type material in a hook and loop type fastening system, that is, the headliner fabric can engage and retain a hook type fastener element attached to a strap, so that splint retaining straps may be attached at different locations of the headliner cover.

In this form of the invention, padding fabric may be applied to opposite sides of a pre-cut splint shape and the fabric may be cut oversized so as to leave fabric fringes extending beyond the edges on each of the opposite sides of the precut splint shape of thermoformable core material. The fringes are then fastened or bonded to each other as by gluing, thermal bonding or sewing, to make a closed seam encompassing all or part of the pre-cut splint-shape, covering and hiding the edges of the thermoformable core sheet.

In any of the aforementioned embodiments where thermoformable laminate has been cut to a splint shape, one or more retaining straps may be fastened to the cut splint shape, for example, by riveting or adhesively bonding the strap to the splint shape.

### BRIEF DESCRIPTION OF THE DRAWINGS

FIG. 1 shows a fragment of splinting laminate according to this invention along with one end of a typical splint retaining strap having a hook type fastener engageable to the padding layer of the laminate;

FIG. 2 shows a typical stock sheet of the novel thermoformable laminate;

FIG. 3 illustrates a typical wrist splint shape cut from the laminate according to this invention;

FIG. 4 shows the wrist splint shape of FIG. 3 bent and formed to a three dimensional configuration suitable for application to a patient's wrist;

FIG. 5 shows the formed wrist shape of FIG. 4 with two splint retaining straps attached to make a complete splint;

FIG. 6 shows the splint of FIG. 5 applied to the wrist of a patient;

FIG. 7 illustrates an alternate form of the wrist splint shape in which padding layers have been applied to a hand splint shape pre-cut from bare thermoformable sheet material and with oversized padding subsequently applied to the pre-cut shape so as to form fringes extending around the edges of the splint shape, the fringes on opposite sides of the splint shape being bonded together to make a seam encompassing the splint shape and covering and sealing the splint edges; and

FIG. 8 is a chart showing manufacturing steps for making different embodiments of the splinting laminate according to this invention.

### DETAILED DESCRIPTION OF THE PREFERRED EMBODIMENTS OF THE INVENTION

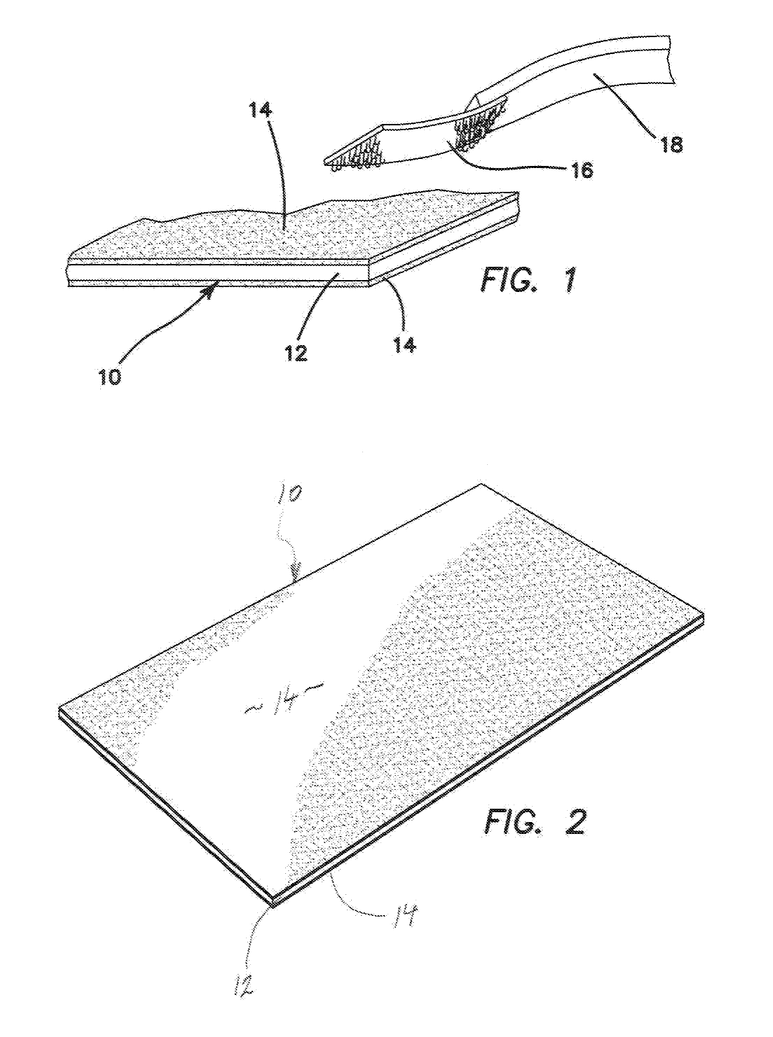

With reference to the accompanying drawings wherein like elements are designated by like numerals, FIG. 1 is an enlarged fragmentary view of the thermoformable splinting laminate, generally designated by numeral 10, according to this invention. Laminate 10 includes a core sheet 12 of normally rigid thermoformable plastic of uniform thickness. Two padding layers 14 are applied to opposite sides of the core sheet 12. The padding material 14 may be bonded or laminated to only one side of the thermoformable core sheet 12 but in most instances it is preferable that the thermoformable plastic 12 be sandwiched or laminated between layers of padding material 14 bonded to the two opposite sides of the thermoformable sheet 12.

The padding layer 14 is chosen to provide a degree of padding or cushioning when applied against the patient's skin. That is, the padding 14 provides a softer, warmer touch compared to the harder, colder feeling of the bare thermoformable plastic core sheet 12, yet without interposing such a thermal obstacle that would excessively slow or prevent heating of the core plastic sheet 12 to a deformable state by external application of heat.

Thermoformable splinting plastics are typically heated to a mechanically deformable state by immersion of the plastic in hot water or by application of a hot air blower or gun for spot molding of specific areas of a splint, so that the material can be bent or formed in three dimensional shape by a therapist as needed to optimally fit the particular patient. The padding material 14 bonded to the thermoformable plastic core sheet 12 is chosen to present a relatively low thermal barrier, so as to allow reasonably fast heating of the covered thermoformable plastic 12.

Suitable thermoformable splinting plastics are known and available, for example, from Patterson Medical in various thicknesses from 1/16″ to as much as ½″. A suitable thickness of splinting plastic is selected according to the degree of support required and can be chosen by the therapist. The smaller thicknesses of these splinting plastics can be cut with appropriate hand scissors without great difficulty for purposes of this invention.

A preferred padding material 14 is of cut polyester fiber applied as loose fiber of uniform length by a flocking process. Flocking application creates a layer of closely arranged fibers, each fiber standing on one end and held to the plastic core sheet 12 by an adhesive, or by application of the fiber while a thin surface layer of the core sheet is in a molten state so that the fiber ends are retained captive when the surface layer solidifies on subsequent cooling. The loose cut fibers are applied in sufficient density so that, to the unaided eye, the resulting appearance is of a substantially smooth surface with a velvet like character. Generally such flocking processes involve application of an adhesive to the core sheet 12 followed by deposition of loose bulk fiber material cut to a uniform short length which may vary depending on the desired thickness, appearance and feel of the finished padding layer 14. In general, the length of the flocking fibers may range from 1/32^{nd} of an inch to ¼ of an inch, or longer.

It has been found that flocked application of padding 14 provides a superior finish having smooth and soft tactile characteristics, an appealingly uniform appearance reminiscent of a velvet fabric, provides a low thermal barrier to heating of the core sheet of thermoformable material, is absorbent of moisture including perspiration while being easy to clean by washing or laundering, and is durable and resistant to wear and immersion in water and subsequent drying. The padded laminate 10 is lightweight and therefore more easily worn and tolerated by patients who are often elderly and enfeebled. Importantly, the padded laminate 10 can be readily cut and shaped in the field using hand tools, such as sturdy scissors, by therapists who can customize splint shapes to the anatomy of individual patients for optimal comfort and effectiveness.

Flocking treatments of this type are well known and flocking services and machinery are commercially available, so that a detailed further description of flocking application and technology is not needed here.

The splinting laminate 10 comprised of thermoformable core sheet 12 and padding material 14 is preferably of a combined thickness which can be cut with hand scissors, to allow a therapist to size, shape and trim a splint shape of such laminate in order to optimize fit for a particular patient on site at a treatment location with simple hand tooling.

In an alternate form of the invention the padding layers 14 consist of textile fabric adhesively laminated to thermoformable plastic core sheet 12. A preferred textile fabric for this purpose is a so-called “headliner” polyester fabric with a 3/16^{th} inch thickness, commonly used for automotive interiors, and available from, for example, onlinefabricstore.net. This fabric has been found to provide a satisfactory degree of padding for patient comfort over the plastic core sheet 12.

The surface of the headliner fabric padding layer functions as one element of a hook and loop type fastener pair. Specifically, it functions as a loop element in a hook-and-loop fastener system. A splint retaining strap such as strap 18 in FIG. 1 can be attached to the splinting laminate 10 by pressing the hook type fastener 16 to the padding layer 14.

This invention is not limited to a particular type of padding material 14 nor to particular methods of attaching, bonding or laminating the same to the thermoplastic core sheet 12. Also the extent of application of the padding material 14 over the thermoformable plastic 12 may vary, parts of which may remain exposed and free of covering.

The laminate material 10 may be sold as flat sheet stock, such as shown in FIG. 2, for cutting and forming by therapists, or may be sold as flat splint blanks pre-shaped for specific uses such as wrist or elbow splints and suitable for trimming and bending by the therapist to a final three dimensional form. The laminate 10 can also be sold already cut and formed into conventional splint shapes ready for application to patients with minimal or no fitting or trimming.

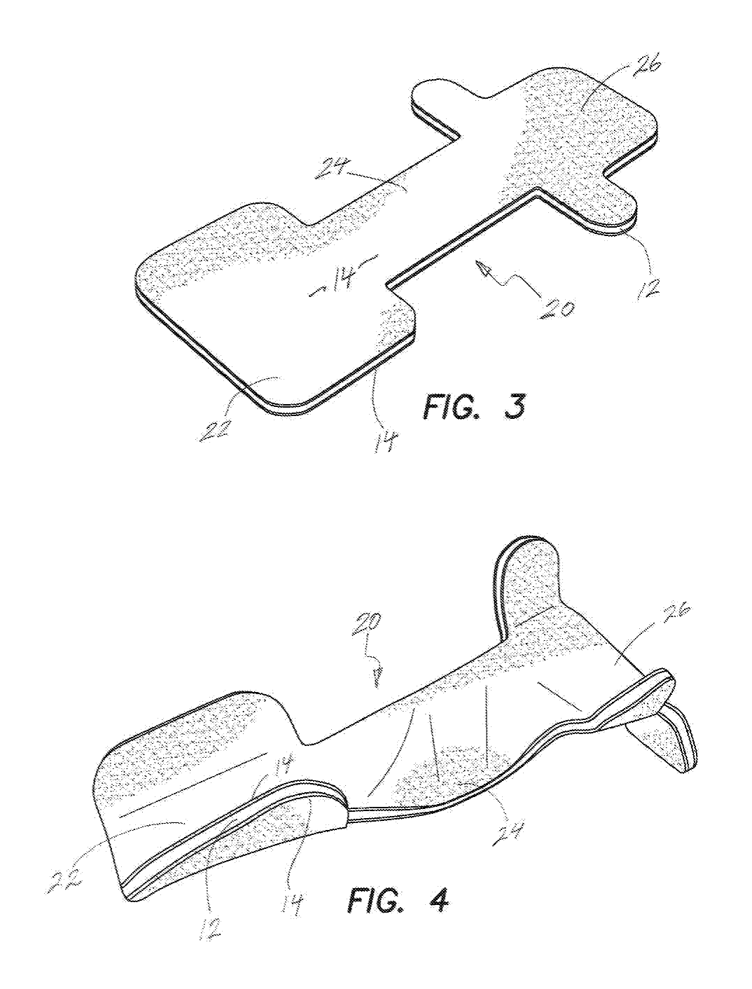

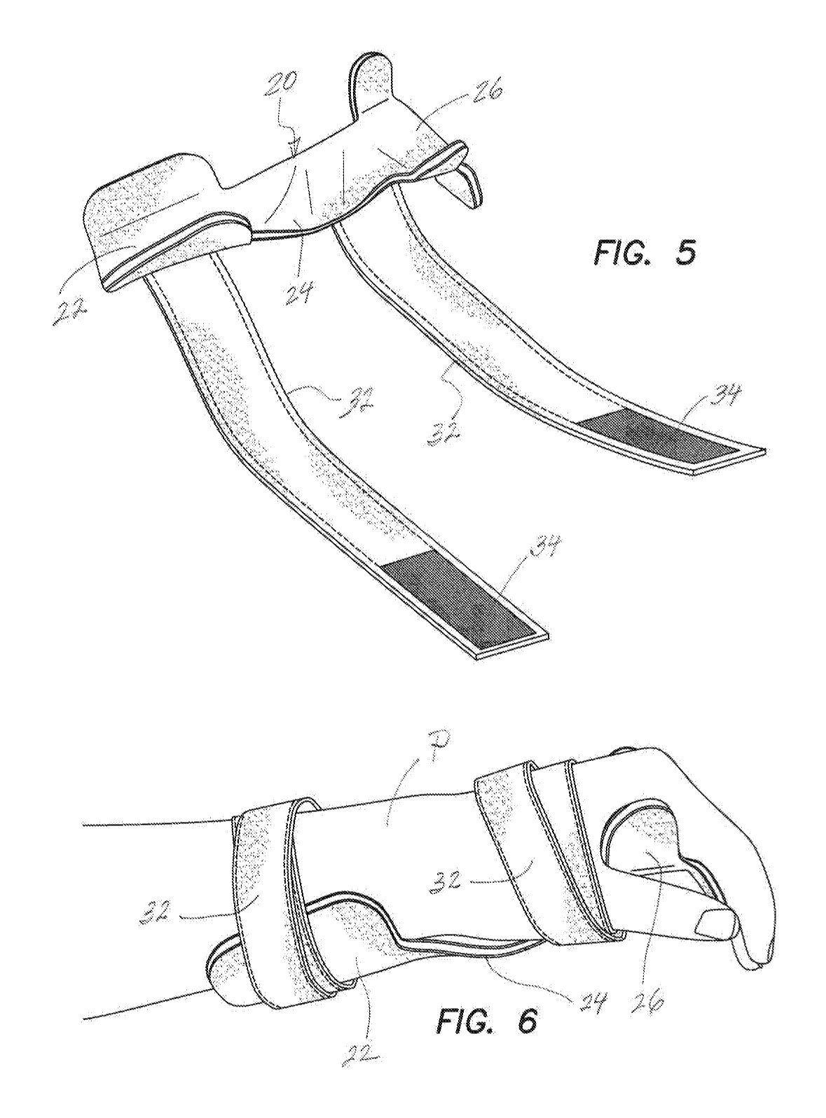

FIG. 3 illustrates a flat splint shape or blank 20 cut from laminate 10. Splint blank 20 is a typical wrist splint shape and in its initial condition is a flat shape, as shown in FIG. 3. Splint blank 20 includes an arm pad 22 connected by a bridge 24 to a hand pad 26. In preparation for use splint blank 20 is bent to an anatomically conforming three dimensional shape such as seen in FIG. 4 by application of heat, e.g. with a hot air blower until the core plastic sheet 12 becomes deformable, followed by application of manual bending force to impart the desired shape to the splint blank 20, after which the resulting formed splint 30 of FIG. 4 is allowed to cool to a substantially stiff, rigid state. The splint 30 is completed by attaching two retaining straps 32 as seen in FIG. 5, one to arm pad 22 and the other strap to hand pad 26. The reverse or outer side of each strap 32 is of a fabric which functions as a loop type element in a hook and loop system, while a hook type element 34 is provided on the other, inner side at the free end of strap 32. The hook element 34 is engageable to the fabric on the reverse side of strap 32, so that when the strap 32 is wound about the patient's limb P in FIG. 6 the end of the strap lies over and against the outside of the wound mid-portion of the strap. Hook element 34 can then be pressed down to make retentive engagement with the underlying strap fabric, thereby to hold the strap 32 against unwinding and to secure the splint 30 in place on patient limb P.

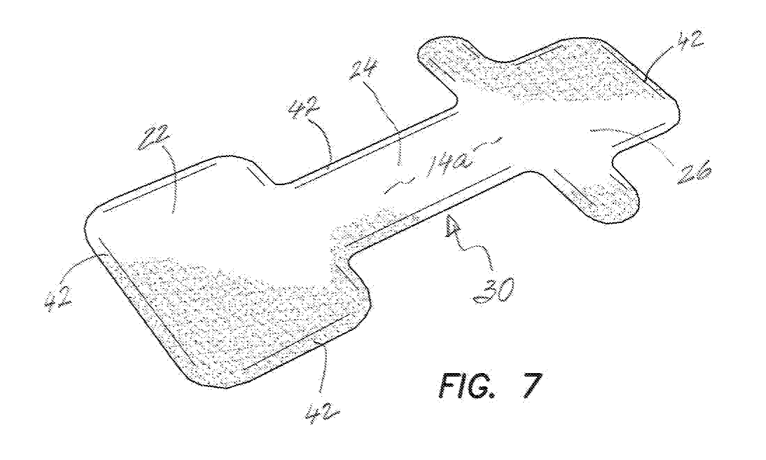

FIG. 7 depicts a hand splint 40 similar to splint 30 of FIGS. 4 through 6, but made by a modified process. Rather than being cut from stock laminate sheet 10, splint 40 is made by first cutting a splint shape from bare thermoformable plastic sheet 12, and then applying padding layers 14a to both sides of the pre-cut plastic splint shape. The padding layers 14a are cut oversized relative to the splint shape so that a border or fringe of padding material extends beyond the edges of the plastic splint shape, preferably along the entire perimeter of the splint shape. The borders of the two padding layers are sealed together as by thermal bonding, gluing, sewing or other appropriate means, to make a closed seam 42 around the splint perimeter. The plastic splint shape is in effect encapsulated by the soft padding material. The closed seam 42 covers the edges of the thermoformable plastic splint shape for greater protection against rubbing and abrasion of skin with the hard edges of the plastic splint shape.

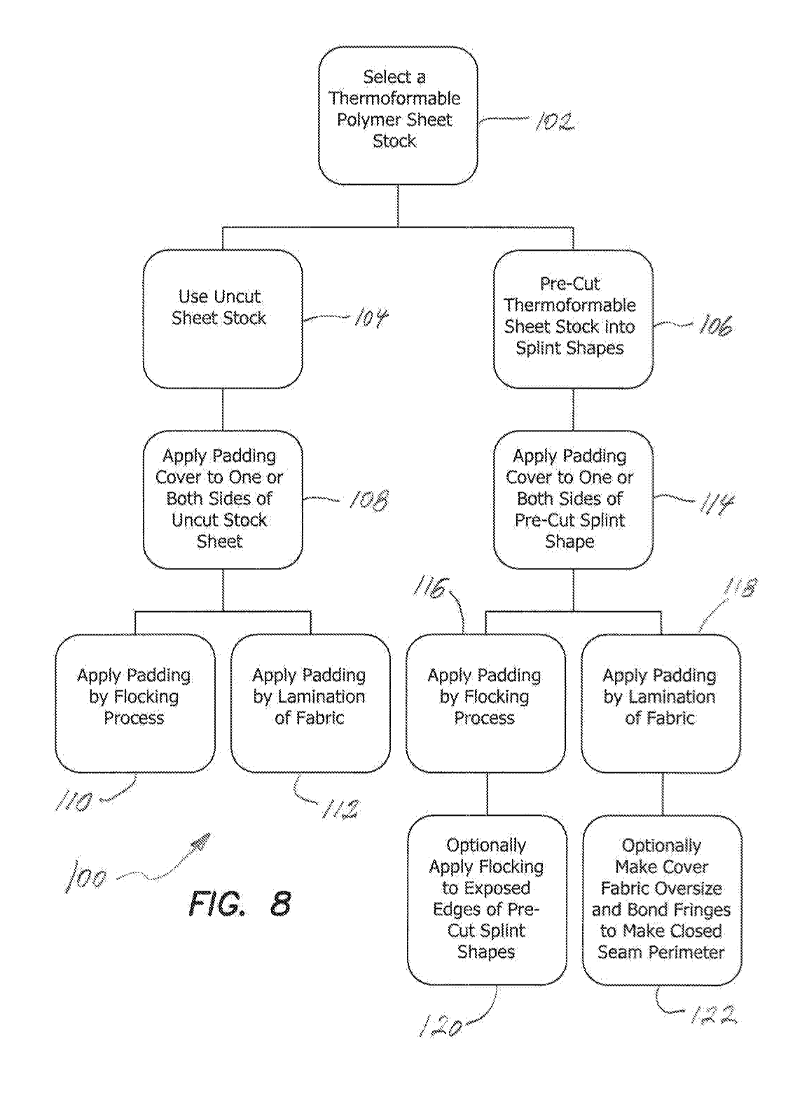

FIG. 8 is a flow chart 100 illustrating the steps for making the different embodiments of the thermoformable splinting laminate 10 and resulting splints described above.

In step 102 a suitable thermoformable splinting plastic is chosen in sheet form. The splinting plastic will typically be acquired from a commercial vendor as sheet stock of given thickness. The splinting plastic may be used in uncut sheet form in step 104, or may be pre-cut into specific splint shapes as flat splint blanks in step 106. The uncut sheet of splinting plastic from step 104 is then laminated by application of a padding to one or both sides of the plastic sheet in step 108. The application of padding may be by flocking application of bulk fiber material in step 110 or alternatively by application of a woven or non-woven fabric, such as headliner fabric, in step 112. The pre-cut flat splint shapes of step 106 are laminated by application of padding to one or both sides of the pre-cut flat splint blanks in step 114. The application of padding in step 114 is either by a flocking process in step 116, or alternatively, by lamination of a woven or non-woven fabric, including the aforementioned headliner fabric, in step 118. From step 116 the flocking application may optionally include and extend to cover the edges of the pre-cut flat splint blanks of splinting plastic in step 120. From step 118 the lamination with fabric may optionally include step 122 of providing fringe borders of padding fabric extending beyond the edges of the splint blank and bonding together the fringes from opposite sides of the splint blanks to make a sealed perimeter seam of padding material covering the edges of the pre-cut splint plastic blank.

From the foregoing it will be seen that this invention provides an easy to use and lightweight laminated splinting material 10 which can be easily cut and formed to provide adjustable and customizable orthopedic support and has an integral cover of padding material for hygiene, comfort and protection from abrasion, such that the padding material 14 can be cut and shaped together with the plastic supporting sheet 12 to make one piece orthopedic splints. The making of custom fitted splints is greatly simplified by this lamination of materials as the splint padding 14, 14a is cut and shaped along with the supporting plastic core sheet 12, greatly simplifying the fitting and application of orthopedic splints as well as eliminating the complications created by separate splint padding covers.

While particular embodiments of the present invention have been described and illustrated for purposes of clarity and example, it should be understood that still further changes, modifications and substitutions will become apparent to those having only ordinary skill in the art without thereby departing from the scope of the following claims.

What is claimed is:

1. An orthopedic splinting laminate composed of a thermoformable splinting sheet material having opposite sides and a covering layer of padding material applied to at least one of said sides for padding the splint for more comfortable application of said splinting sheet material to the limb of a patient.

2. The laminate of claim 1 wherein said covering layer is applied to said thermoformable splinting sheet material by a fiber flocking process.

3. The laminate of claim 1 wherein said covering layer is adapted for making retentive engagement with a hook or loop type fastener such that a retaining strap provided with a said hook or loop type fastener may be attached at any desired location of a splint cut from said laminate.

4. The laminate of claim 1 wherein said covering layer is applied to cover one or both sides of an uncut stock sheet of said thermoformable splinting sheet material so that splint shaped blanks may be subsequently cut from said stock sheet.

5. The laminate of claim 1 wherein said covering layer is applied to a pre-cut splint shape cut from a stock sheet of said thermoformable splinting sheet material.

6. The laminate of claim 5 wherein said covering layer is applied to said pre-cut splint shape by a fiber flocking process to one or both sides of said splint shape.

7. The laminate of claim 6 wherein said flocking is also applied to some or all exposed edges between opposite sides of said pre-cut splint shape.

8. The laminate of claim 5 wherein said padding layer is a woven or non-woven fabric adhesively applied to said thermoformable splinting sheet material.

9. The laminate of claim 8 wherein said fabric is applied to opposite sides of said pre-cut splint shape and said fabric is cut oversize to said pre-cut splint shape thereby to form fringes on said opposite sides, said fringes being fastened to each other to make a seam about said pre-cut splint-shape.

10. An orthopedic splint made by cutting the laminate of claim 1 to a desired splint shape and fastening one or more retaining straps to said cut splint shape.

11. The orthopedic splint of claim 10 wherein said retaining straps are fastened by riveting to said cut splint shape.

12. The orthopedic splint of claim 10 wherein said retaining straps are fastened by adhesively bonding to said cut splint shape.

13. The orthopedic splinting laminate of claim 1 wherein said thermoformable sheet material is normally rigid at room temperature and is deformable for shaping manually to a three dimensional shape following heating as by immersion in hot water or application of heated air as by a heat gun type device.

14. The orthopedic splinting laminate of claim 1 wherein said padding cover material is relatively thin and heat permeable such that said thermoformable splinting material is heated to a manually bendable state within a relatively short time following either immersion in hot water or application of a stream of hot air delivered by a conventional hand held electrically powered hot air blower gun.

15. The orthopedic splinting laminate of claim 1 wherein said padding cover material provides a hygienic and relatively comfortable surface against the skin of a patient.
